(19) United States
(12) Patent Application Publication
(10) Pub. No.: US 2017/0167096 A1
(43) Pub. Date: Jun. 15, 2017

(54) Inflatable Article with Reduced Stress Concentrations

(71) Applicant: Henry Obermeyer, Wellington, CO (US)

(72) Inventor: Henry Obermeyer, Wellington, CO (US)

(21) Appl. No.: 15/024,173

(22) PCT Filed: Sep. 23, 2014
(86) PCT No.: PCT/US14/57076

§ 371 Date: Jul. 20, 2016

Related U.S. Application Data
(60) Provisional application No. 61/881,447, filed on Sep. 23, 2013.

(51) Int. Cl. E02B 7/20; E21F 17/107; A61F 2/44; B63C 9/08; F16G 1/06; E02B 3/10; B63B 35/73

(52) U.S. Cl. CPC E02B 7/20; E02B 3/102; E21F 17/107; B63B 35/73; B63C 9/08; F16G 1/06; A61F 2/441; A61F 2/442; A61F 2002/4475

(57) ABSTRACT

The present invention relates to collapsible hoses and inflatable structures such as pneumatic actuators for water control gates that are manufactured on and incorporate into their structure a flexible internal mandrel with rounded edges. One face of the mandrel and all of its rounded edges separate from the inflatable membrane during pressurization, while the remaining face of the flexible mandrel remains bonded to the remaining face of the inflatable envelope. Sharp internal edges (in the deflated condition) and their associated stress concentrations (in the inflated condition) are thus eliminated.

This application is the United States National Stage of International Application No. PCT/US2014/057076, filed Sep. 23, 2014, which claims benefit of and priority to U.S. Provisional Application No. 61/881,447 filed Sep. 23, 2013, each of said applications hereby incorporated herein in its entirety by reference.

### BACKGROUND OF THE INVENTION

1. Field of Invention

The present invention relates to inflatable articles, such as collapsible hoses, ship launching bladders, inflatable structures such as pneumatic actuators for bottom hinged inflation actuated water control gates, inflatable rubber dams, inflatable seals, and the like, each with reduced stress concentrations (in comparison to prior art articles of similar function) at the edges which unfold and fold during inflation and deflation, respectively.

The present invention additionally relates to inflatable articles such as hoses wherein it is desirable that residual fluid within the deflated article be minimized.

An example of a hose wherein residual fluid in the otherwise empty/deflated/depressurized condition is undesirable is hose used for petroleum transfer at sea, in conjunction with which residual oil or fuel left in a hose may result in an environmentally hazardous or flammable spill. Another example is a hose wherein expensive residual fluid would be wasted or lost. Conversely, an otherwise empty prior art hose, the open ends of which may be exposed to seawater during underwater connection or reconnection operations, for example, may become undesirably, but unavoidably, partially filled with seawater which naturally occupies voids along the interior edges of the otherwise flattened hose.

A further example of the field of application of the present invention is hose or bags used for blood transfusion or blood treatment, in conjunction with which blood left in a hose or bag may be wasted, while blood left stagnant for a short period of time in the voids of a prior art hose may clot.

A further example of the field of the invention is hose which must be flattened and coiled for storage or transport. In this case a hose of uniform flattened thickness may coil and uncoil more readily and more reliably than a hose of varying thickness across its width. Typical collapsible hoses of prior art exhibit thickened and not fully collapsed edges even when the middle of the flattened hose is fully collapsed. The durability of a hose that is repeatedly coiled and uncoiled in many instances depends on the minimization of stress concentrations along the edges of the hose which is a primary subject of this invention. It is also desirable that a minimum amount of residual fluid remain in a hose when it is emptied and coiled. The minimization of residual fluid within a collapsed or emptied hose is a further object of this invention.

A further example of the field of the invention is that of hoses which may benefit which must be emptied or deflated under conditions of extraordinary external pressure, such as when used in sub-sea service.

Many applications require hose with multiple plies of reinforcing cord. Such hoses have heretofore been unable to be cured in a flattened configuration because of the severe stress concentrations that would result upon internal pressurization of the hose. Curing (or vulcanizing) such hoses on a mandrel limits the length of each hose segment and necessitates the use of connections between individual mandrel cured lengths of hose and results in hoses which, in general, cannot be flattened. The reduced stress concentration hose of the present invention enables the manufacture of lengths, limited only by transportation constraints, of hose with multiple plies of reinforcement. Transportation of hose in accordance with the present invention is all the less constrained because hose in accordance with the present invention may be fully flattened and tightly spooled. Example applications for high pressure flattenable hose include land based, marine and sub-sea crude oil transfer, dredging, effluent transport, water transport, fire fighting, compressed air supply, etc.

Inflatable structures, which are used to raise and lower other objects, are another example within the field of the invention. Specific examples include inflatable bladders or actuators used to actuate water control gates, bladders behind the gates that control water flow, inflatable seals to prevent flow in tunnels, among other examples.

Still other inflatable objects within the field of the invention include flotation devices used for safety or recreation, as well as water storage bladders such as those used in specialized back packs. Eliminating residual fluid prior to storage results in improved hygiene. Related items include flotation devices for recreation or water safety, such as air mattresses and inflatable pools and pool toys.

Another application of the invention is for inflatable actuators for water control or traffic control gates which must support traffic loads transmitted through the gate panel when in the deflated configuration. In this case, the rubber mandrel inside of the deflated inflatable actuator can serve to transmit the loads to the underlying foundation.

2. Description of Related Art

Pneumatically operated water control gates are well known. Prior art includes U.S. Pat. No. 4,780,024 to Obermeyer et al; U.S. Pat. No. 5,092,707 to Henry K. Obermeyer; U.S. Pat. No. 5,538,360 to Henry K. Obermeyer; U.S. Pat. No. 5,642,963 to Henry K. Obermeyer; U.S. Pat. No. 5,709,502 to Henry K. Obermeyer; U.S. Pat. No. 5,713,699 to Obermeyer et al. and U.S. Pat. No. 7,114,879 82 to Henry K. Obermeyer.

Hoses of prior art are generally molded in a round configuration. The degree of tendency to remain round during use and storage is a function of hose construction which depends on intended use and internal and external pressures. Suction hoses, such as those associated with household vacuum cleaners and sewage pumps, operate under conditions of higher external pressure than internal pressure and must therefore be stiffened against undesirable collapse during use. Such stiffened hoses fall outside of the field of the present invention. Hoses intended for very low pressure may be made very thin and therefore tend to collapse easily, rendering the hose easier to store and spool, as is the case with fabric fire hose and large diameter polyethylene film irrigation tubing. Such hoses are generally not subjected to extreme external pressures and are therefore generally not forced to totally collapse at their edges. The absence of noxious fluid remaining along the internal edges of the collapsed hose in combination with the absence of severe stresses along the outside of the collapsed hose place these particular applications generally outside of the scope of the present invention.

Certain hose applications might be best served with a hose capable of both high operating pressures and full collapse under high external pressure without undue stress concentrations along the margins of the hose which must fold and unfold during depressurization and pressurization, respectively. It is this combination of relatively high pressure rating, the ability to fully inflate without the development of high interior tensile stresses along the erstwhile edges of the hose, along with the ability to fully collapse without the development of tensile stresses along the exterior edges of the hose that is a primary object of the hose embodiment of the present invention.

Hoses of prior art, such as fabric reinforced fire hoses, tend to collapse to a configuration that is nearly flattened, but which exhibits along its edges residual fluid filled voids. The residual fluid may be very difficult to remove from the hose and its existence is in almost all applications undesirable because of the commercial value of the fluid, the fact that the fluid may later freeze as in the case of water, or the fluid may cause deleterious pollution as in the case of a crude oil transfer hose at sea. Lacking an internal mandrel, or stress relief insert, the edges of such hoses undergo severe strain between deflated (depressurized) and inflated (pressurized) states. The location of the stresses resulting from this strain depends on the configuration in which the hose was molded, the most common configurations being fully flat or fully circular.

Pneumatically operated gates are generally designed such that the inflatable air bladder resists the forces of the water to be controlled. This requires a bladder with sufficient strength and size to resist the forces of the water on the gate panel.

Pneumatically operated gates generally are inclined in the direction of water flow. This facilitates the passage of water borne debris and ice but also facilitates the passage of waves up over the gate, a characteristic that is a drawback in the case of a gate one of the purposes of which is to prevent damage by waves.

### 3. SUMMARY OF INVENTION

There are several categories of embodiments of the present invention. One of the categories is hose. Another category is inflatable actuators for water control gates. Yet another category is inflatable seals. A broad range of other categories exist as well which are too numerous to list individually. The common attributes of the various categories will however be discussed in detail in this specification. An internal elastomeric mandrel is a defining aspect of many of the embodiments of this invention. Such a mandrel may serve one or more of a plurality of purposes. One on the purposes of the mandrel is to serve as a mold surface with marginal edges of a specified radius which establish the edge radius along the interior marginal edges of the folded or collapsed inflatable article. In the absence of such a mold surface, the interior marginal edges tend to become sharp during molding or curing. Such sharp interior marginal edges become highly stressed when the article is inflated. In the case of multi-layer structures comprised of an inner liner, one or more fiber reinforced layers, and an outer cover, the inner liner in subject to the highest strains. Although these highly stressed interior edges may not fail during a single inflation of short duration, multiple inflation cycles may cause fatigue failure and extended periods of inflation may cause stress corrosion cracking such as may be induced by ozone in air or chlorine in water, for example. Failure of the inner liner may allow leakage of fluid or gas into the interstices between reinforcing cord strands. This may cause damage to the reinforcing cord strands and will likely cause an increase in inter-carcass pressure, which may in turn lead to de-lamination. In the case of inflatable articles with multiple layers of reinforcement, the transition during inflation from a near zero edge radius to a the much larger radius characteristic of the fully inflated article results in unequal load sharing between the inner outer plies of reinforcement and may result, for example, in only the outermost pair of plies carrying no tensile load whatsoever.

In accordance with prior art an internal edge radius of specified dimension may be obtained by one of several methods. By one method, an internal mandrel or tool is molded into the article and later removed from the cured inflatable article. Removal requires an opening elsewhere in the inflatable article. The requirement for an opening severely restricts the types of articles for which this method is useful.

By a second method, a water soluble mandrel may be used. Water soluble mandrels tend to be expensive, are fragile and easily broken, and require significant time and expense for removal.

By a third method, stress relief inserts may be positioned along the marginal edges of the inflatable article during manufacture as disclosed in U.S. Pat. No. 7,114,879 B2 to Henry K. Obermeyer. Stress relief inserts usefully reduce stresses that result from inflation and prevent outer layer stresses that would result from external crushing forces, but have several important limitations relative to the present invention as follows: Firstly, the stress relief inserts provide by themselves no dimensional control during manufacture of the inflatable article. Secondly, the resulting inflatable article is inherently of non-uniform thickness. The non-uniform thickness greatly complicates the manufacture of tooling or molds for the exterior surfaces of the inflatable article. The non-uniform thickness also makes use of the inflatable article as a weight bearing pad, as in the case of a traffic bearing water control gate, much less convenient. In the case of a long hose to be spooled, the uniform thickness facilitated by the present invention facilitates the spooling of the hose while also causing the process of spooling to create a uniform internal pressure between opposing elastomeric hose faces. The pressure between opposing internal hose surfaces acts to expel any residual liquid from the hose during spooling. The presence of residual fluid within a spooled hose is undesirable in many instances. In the case of water, residual water in a hose may freeze and prevent or delay subsequent unspooling of the hose. In the case of hoses used for more than one fluid, residual fluid from one use may contaminate another fluid during a subsequent use.

It is an object of one aspect of this invention to provide a hose that may be molded in a flattened shape and then repeatedly pressurized and depressurized with minimal stress concentrations. As a result of having been molded in a generally flattened configuration around an internal elastomeric mandrel with rounded edges, the hose tends to readily collapse to this flattened and void-free configuration when deflated. During collapse, fluid along the edges of the bladder, which would remain in the case of a conventional flattenable hose, is displaced by the rounded edges of the elastomeric mandrel, allowing the fluid to exit the hose rather than remaining in the hose along each edge.

In accordance with a further object of one embodiment of this invention, a flattened hose incorporating one or more stress reduction elements may be cured in a spooled configuration in an autoclave, for example.

In accordance with a further aspect of this invention, a hose cured in its spooled configuration may be transported to its point of use as cured without unspooling.

In accordance with a further aspect of this invention a hose cured in its flattened configuration may be transported to its point of use with the same spool flanges and spool core as those used during the curing.

In accordance with one embodiment of this invention, ship launching or recovery air bags may be manufactured in a manner similar to the above described hose embodiment, but with closed, and preferably tapered ends. Squared-off ends in the as-manufactured and deflated condition tend to protrude like the corners of a pillow when inflated and thus interfere with the rolling movement required for ship launching or recovery. The use of ends tapered in their deflated configuration prevents the occurrence of protruding inflated corners which may interfere with rolling.

In accordance with one embodiment of this invention, an attachment means such as an elastomeric wedge assembly may be incorporated into the inflatable article of this invention. Such an elastomeric wedge assembly is particularly useful for securing a pneumatic actuator within a water control gate assembly.

In accordance with one embodiment of this invention a stress reducing element may be cured prior to incorporation into the structure of the inflatable article as a whole, and used during construction of the inflatable article as a whole as a mandrel upon which the subsequent layers may be built. The stress reducing element thus serving as an elastomeric mandrel that serves initially as a tool upon which the article may be built, serves during curing as an internal mold that usefully provides a defined inside edge radius to the inflatable article, and serves during use of the article to prevent crushing of the edges of the article under high external pressure while also displacing otherwise residual fluid during emptying of the article.

In accordance with a further aspect of this invention, an air fitting may be molded into a rubber mandrel, which is in turn molded into an inflatable article. In accordance with a further aspect of this invention, the rubber mandrel around which the bladder is built may serve to transfer loads through the deflated air bladder. An example of such an embodiment is a vehicular traffic bearing storm water control gate wherein the forces imparted by vehicle tires to the gate panels are in turn imparted to spacer elements (if used), then in turn imparted to the upper bladder membrane, then in turn imparted to the rubber mandrel, then in turn imparted to the lower bladder membrane, then in turn imparted to the underlying (typically concrete) foundation. Bending moments within the gate panels due to vehicle weight are thus minimized, providing longer gate panel life and allowing the use of a lighter and thinner assembly. A thin gate assembly is particularly important for gates installed on an existing generally planar street or parking garage ramp, for example, where a thick gate assembly might result in insufficient under-vehicle clearance and might also result in damage to vehicles or to the gate system.

In accordance with a further aspect of the invention, the rubber mandrel may serve to reduce local crushing loads imparted to the deflated article of this invention. For example, a rubber dam built in accordance with the present invention would incur less damage due to impact of a large stone than a similar rubber dam lacking the internal rubber mandrel.

In accordance with a further aspect of the invention, a hose constructed in accordance with the present invention may be used in conjunction with peristaltic pumps. The void free configuration of the flattened hose reduces or eliminates back-flow leakage along the folded edges of the hose and thereby provides superior pumping efficiency. Additionally, during the repeated hose inflation and collapse associated with the function of peristaltic pumps, the reduction of cyclic stresses enabled by the hose in accordance with the present invention provides or an extended hose life, higher hose reliability, and peristaltic pump system reliability. The reliability of peristaltic pump hoses is of utmost importance in medical applications and for the pumping of toxic or flammable fluids. Additionally, peristaltic pump hose in accordance with the present invention may be designed for higher pressure than peristaltic pump hose of prior art. The higher hose pressure rating allows, in turn, a higher peristaltic pump pressure rating and thus enables the use of peristaltic pumps in applications for which they could heretofore not generate enough pressure. Peristaltic pump hose has heretofore been most commonly made by the extrusion of soft elastomeric materials without reinforcing cord. Peristaltic pump hose in accordance with the present invention may be reinforced in accordance with high pressure hose practice utilizing, for example, nylon, polyester, aramid, or steel wire reinforcement, for example. The erstwhile inability of high pressure reinforced hose to flatten in the manner required for efficient peristaltic pump operation is overcome in accordance with the present invention by the presence of a stress relief insert within the hose of sufficient thickness and edge radius dimension to seal against the interior of the hose that is flattened not completely but to a degree consistent with its ability to repeatedly deform without failure over the course of an acceptable operating life as the hose component of a peristaltic pump.

In accordance with a further aspect of this invention a hose subject to external flows substantially normal to the hose axis, as in the case of a marine petroleum transfer hose subject to sea currents for example, may incorporate a surface texture selected to induce vortex generation of a scale that energizes the boundary layer and thereby minimizes flow separation and thereby also drag and von Karman vortex shedding and associated vibration. Example textures that may be used for this purpose include repeating chevron patterns, shark skin texture, as well as textures used for racing swimwear and the like.

In accordance with a further aspect of this invention, an inflatable article, such as a water control gate inflatable actuator, may be built around the combination of an elastomeric mandrel and a removable mandrel, wherein the removable mandrel creates an inflation fluid passageway into and, optionally, through, the inflatable article. Such a configuration is particularly useful in conjunction with motor vehicle supporting water control gates which must often be installed on and secured to an existing street or parking garage ramp, for example. In this instance, an inflation fluid passageway through the inflatable actuator eliminates the need to establish passageways for hoses and the like in the underlying foundation. A first inflatable actuator may be connected to a controlled inflation fluid, air for example, supply one edge. Adjoining inflatable actuators may be connected in daisy-chain fashion to each other in order to establish a common inflation manifold without the need to cut into or potentially structurally compromise the pre-existing underlying foundation. In the case of a water control gate actuator, such a removable mandrel may advantageously adjoin the elastomeric mandrel along its secured-to-foundation edge. It this case the defined internal radius of the edge built around a portion of the removal mandrel is established directly by the removable mandrel itself.

It is an object of one aspect of this invention to provide a water control gate that can readily resist the forces caused by storm surges and waves without the need for a large high pressure air bladder.

It is a further object of this invention to present to oncoming waves a surface inclined to as to turn back waves into the direction from which they came.

It is a further object of this invention to provide a generally flat upper surface suitable and safe for pedestrian and vehicular traffic.

It is a further object of this invention to provide a load path down through the gate structure to the foundation, which load path may include the deflated actuating bladder and may include the collapsed restraining (position limiting) members.

It is a further object in certain embodiments of this invention to provide a protective shield for the air bladders, which shield may include the restraining members.

It is a further object of this invention to provide an inflatable bladder that is not subject to high stress concentrations as a result of transitioning from its as-manufactured shape to its inflated shape.

In accordance with a further object of this invention, a rubber mandrel may be provided that is comprised of two parts, one part being adhered to one interior surface and second part being adhered to a second interior surface. Deflated-radius conforming features may be incorporated into either part singly, or may be split and incorporated into both parts. In this manner the benefits that the extra thickness to the overall rubber structure that is provided by the rubber mandrel is shared by both membranes. These benefits may include, for example, reduced permeability to the inflation medium, reduced leakage at points of penetration due to damage by bullets or sharp objects, for example. The proportion of the mandrel attached to either surface, and the shape of the deflated radius conforming features may be varied at will without losing the fundamental benefit of this invention.

### 4. BRIEF DESCRIPTION OF THE DRAWINGS

Note that the following drawings relate to one or more embodiments only and are not in any way to limit the invention, embodiments thereof, claims, or elements thereof. Other objects, advantages and capabilities of the present invention will become apparent as the description proceeds taken in conjunction with the following drawings in which:

FIG. 1 illustrates an inflated hose in accordance with the prior art.

FIG. 2a illustrates an inflated hose of prior art.

FIG. 2b illustrates the hose of FIG. 2a in its deflated configuration.

FIG. 3a illustrates a deflated hose of prior art.

FIG. 3b illustrates an edge detail of the hose of FIG. 3a.

FIGS. 4a, 4b, 4c and 4d illustrate a hose in accordance with the present invention.

FIG. 5 illustrates a hose in accordance with the present invention.

FIG. 6 illustrates a hose in accordance with prior art.

FIG. 7 illustrates a hose in accordance with the present invention.

FIGS. 8, 9, 10, 11, 12a, 12b, 13, 13b illustrates a drive-over water control gate in accordance with the present invention.

FIGS. 14 and 15 illustrate a drive over wave barrier gate in accordance with the present invention.

FIGS. 16 and 17 illustrate the bladder actuator of a drive-over gate actuator.

FIGS. 18a and 18b illustrate a hose in accordance with the present invention.

FIG. 19 illustrates a peristaltic pump hose in accordance with the present invention.

FIG. 19 illustrates another peristaltic pump hose in accordance with the present invention.

FIG. 21 illustrates a hose in accordance with the present invention.

FIGS. 22a, 22b, 23a, and 23b illustrate inflatable bladders in accordance with the present invention.

FIGS. 24, 25, 26 and 27 illustrate edges details of inflatable articles in accordance with the present invention.

FIGS. 28a and 28b illustrate an inflatable seal in accordance with the present invention.

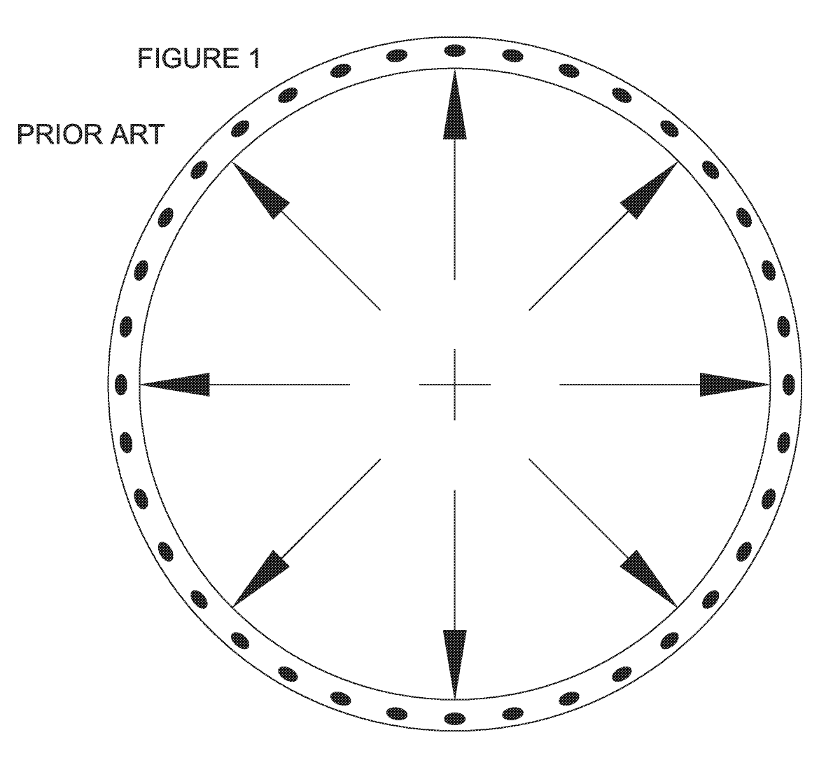

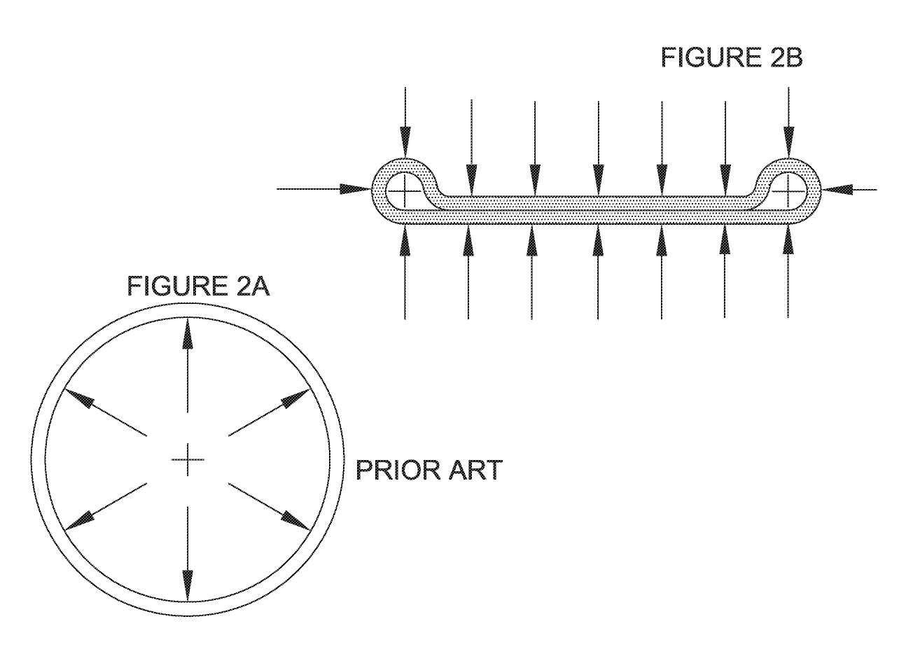

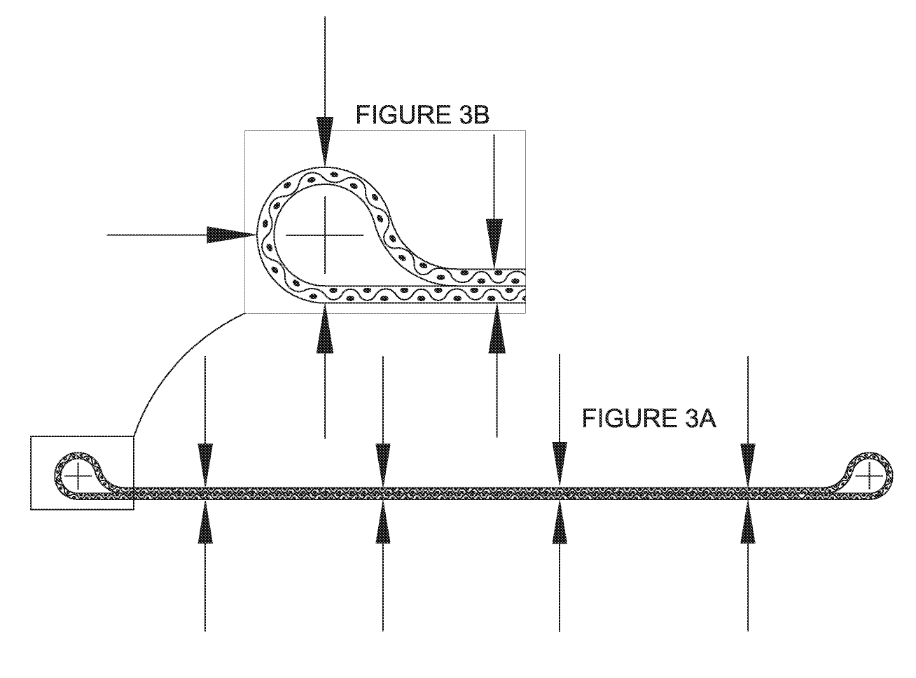

### 5. DETAILED DESCRIPTION OF THE PREFERRED EMBODIMENTS

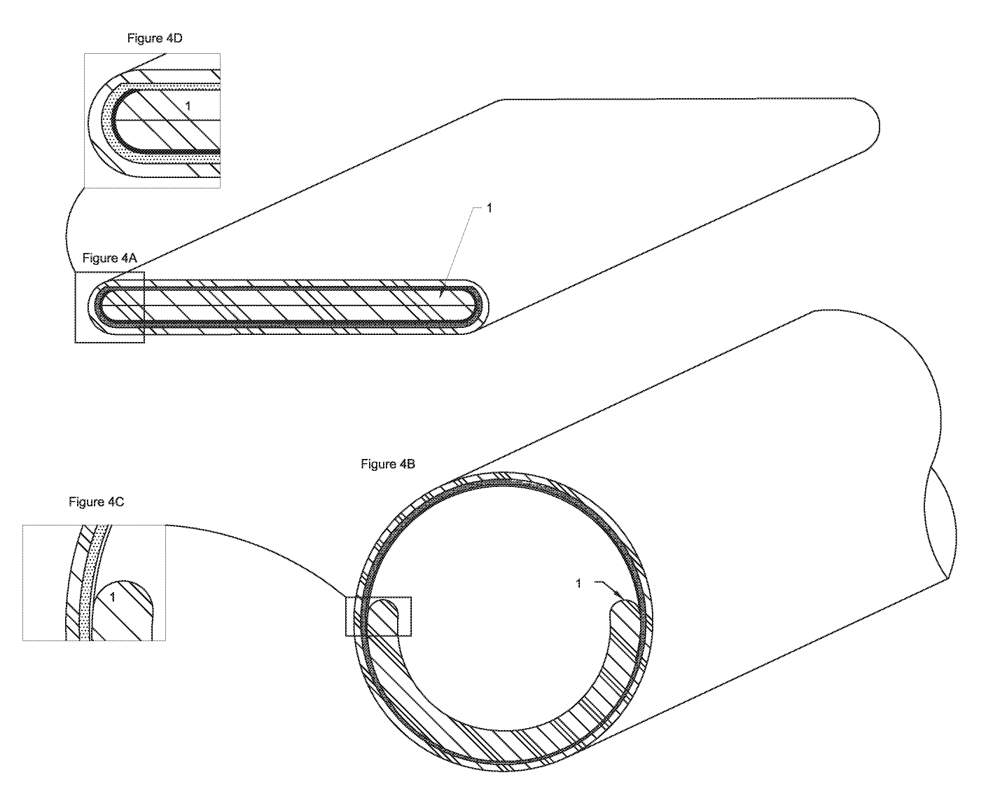

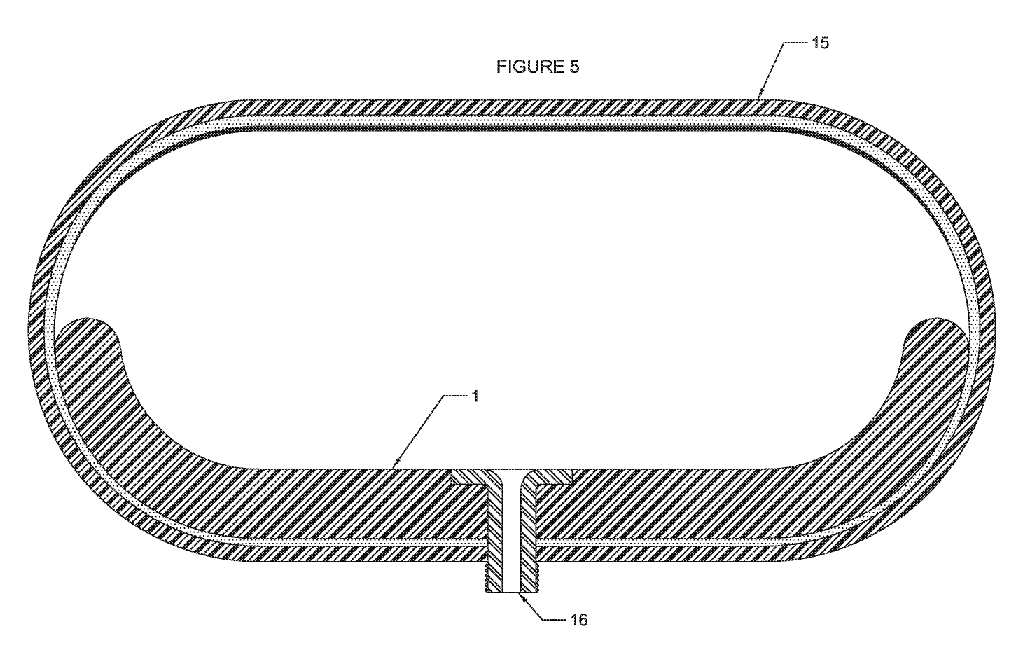

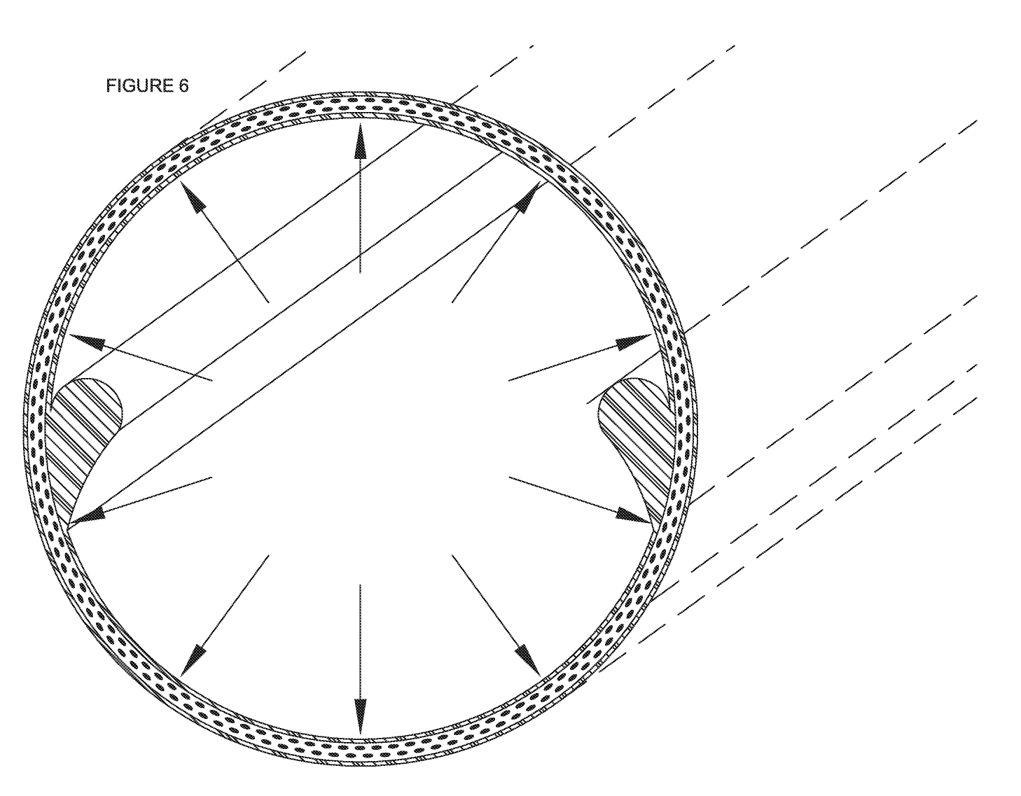

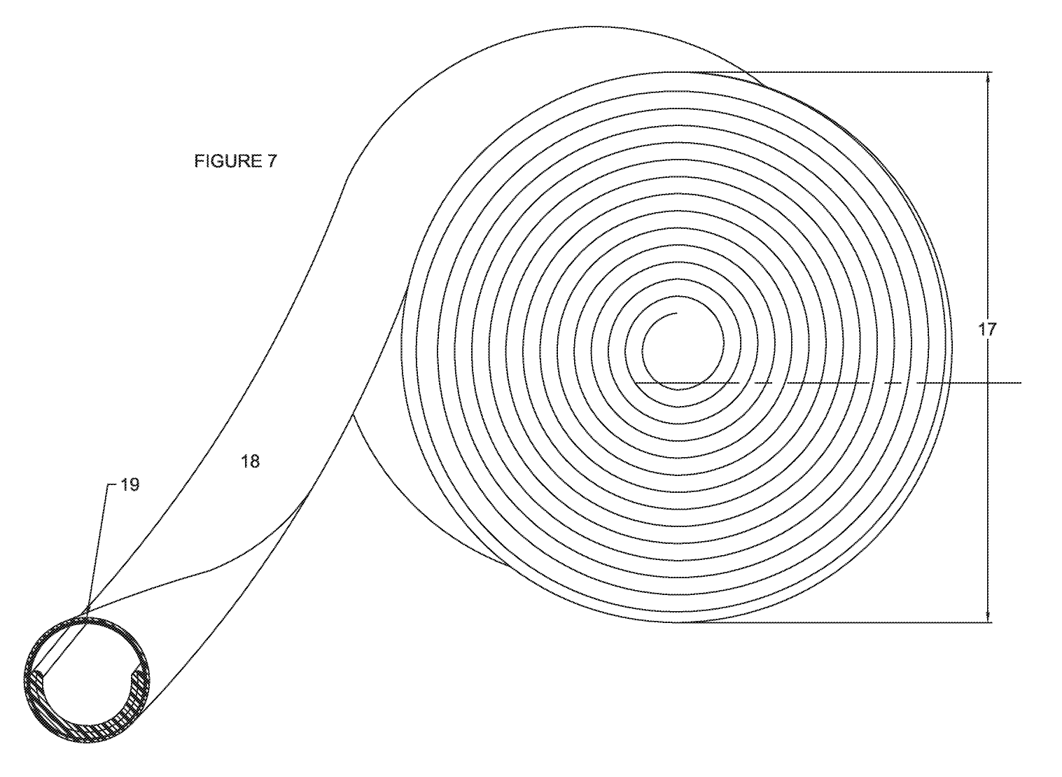

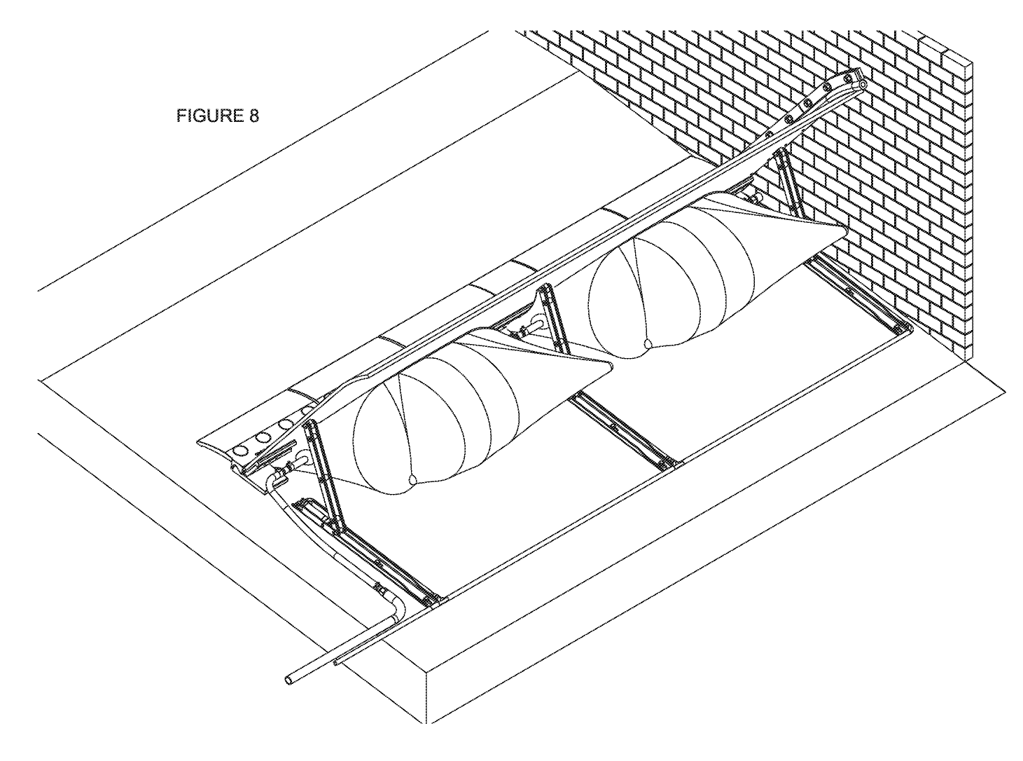

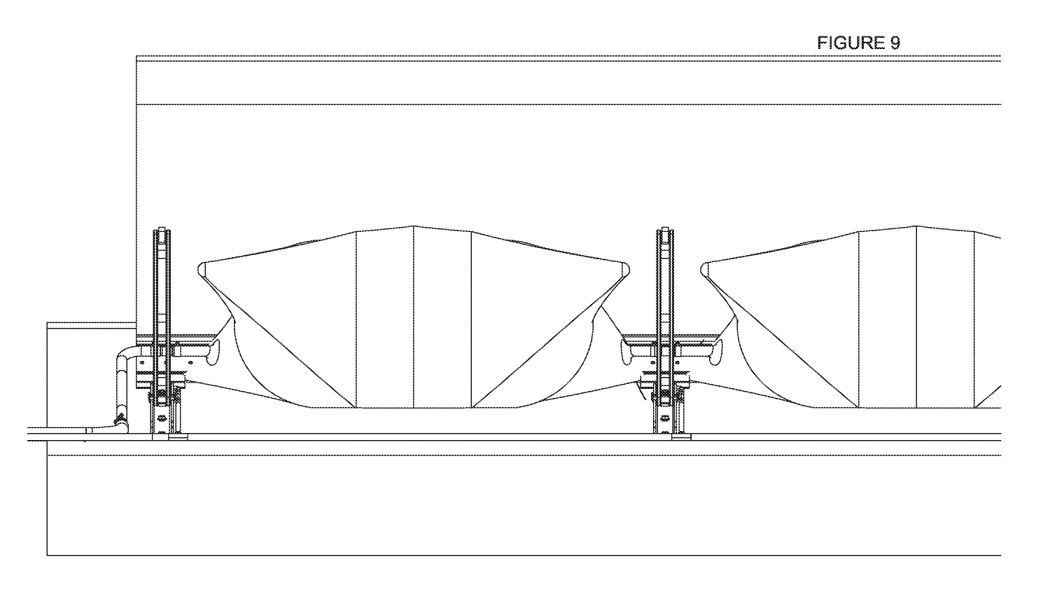

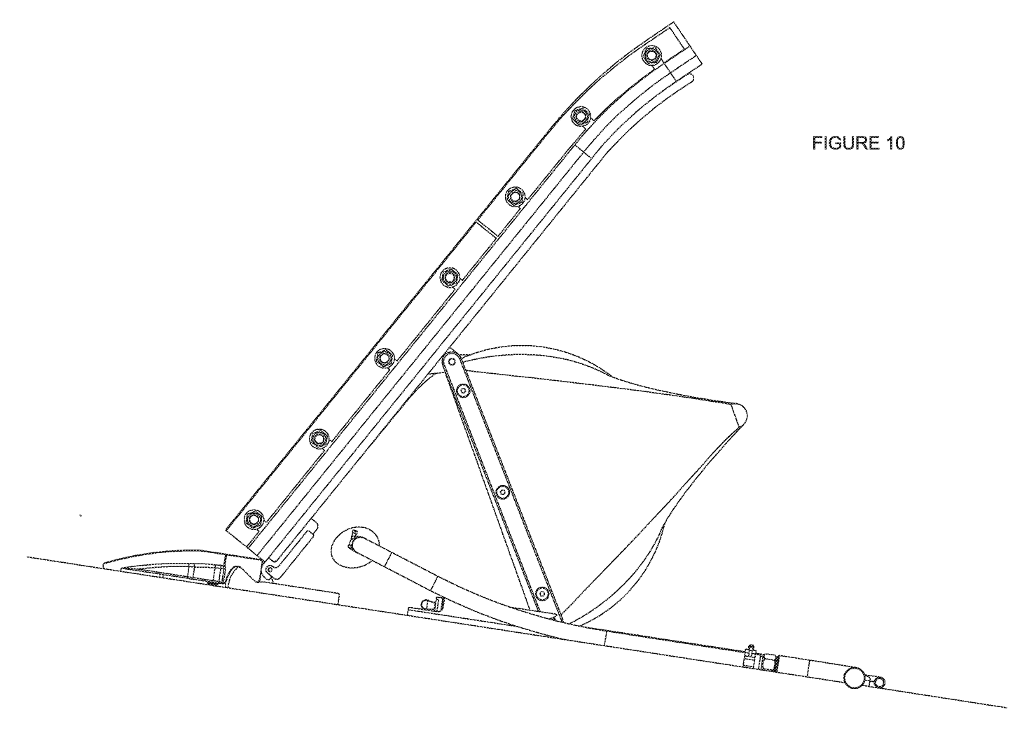

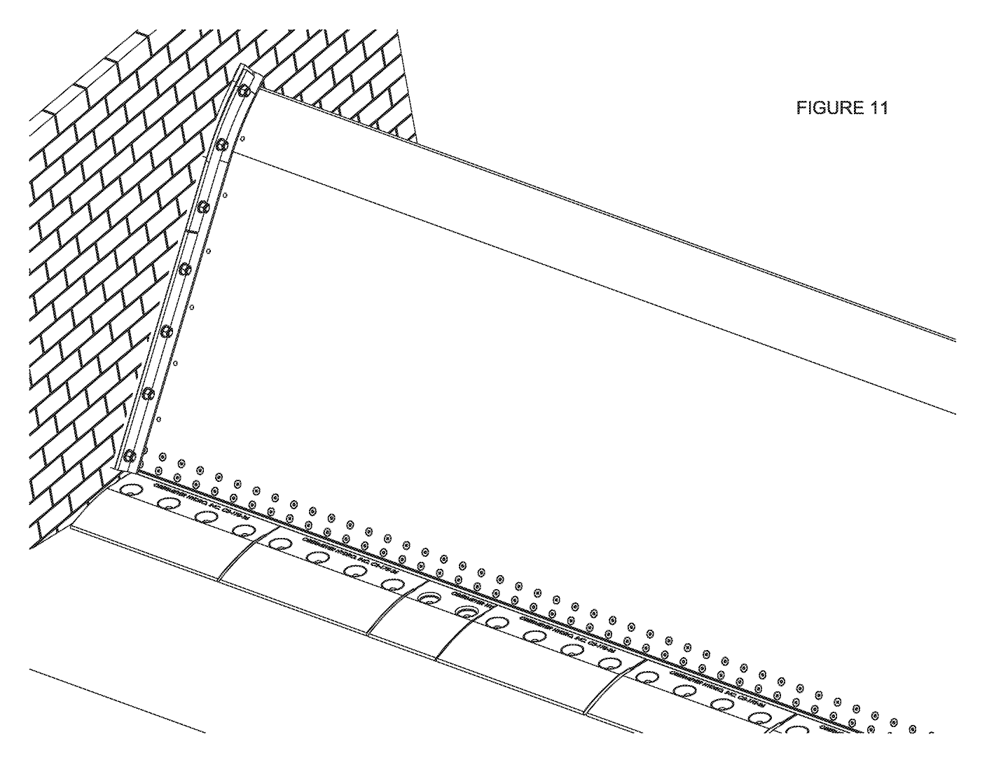

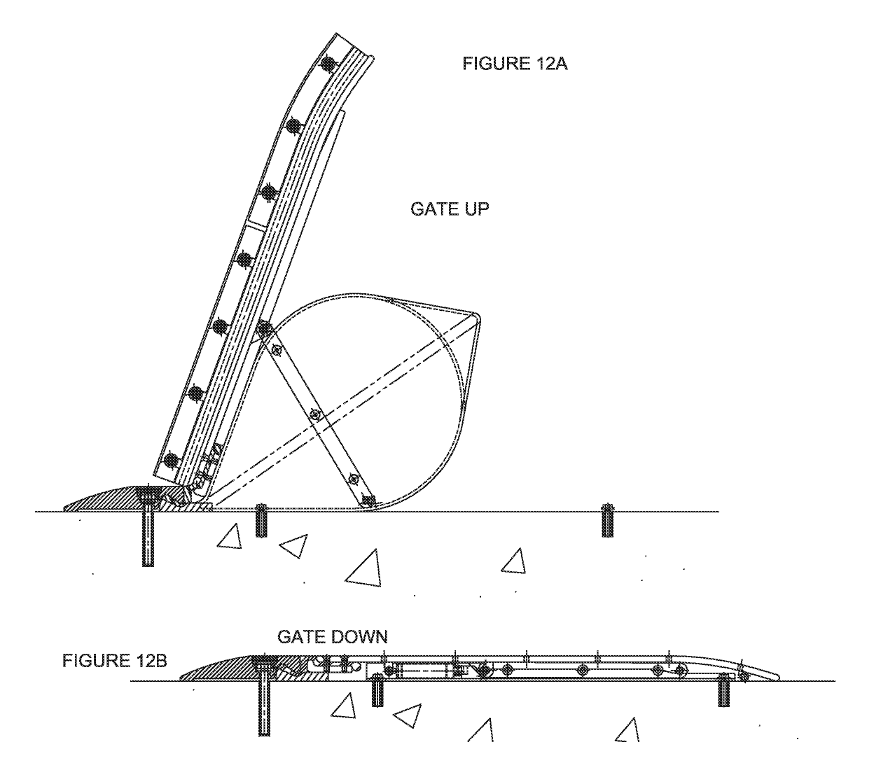

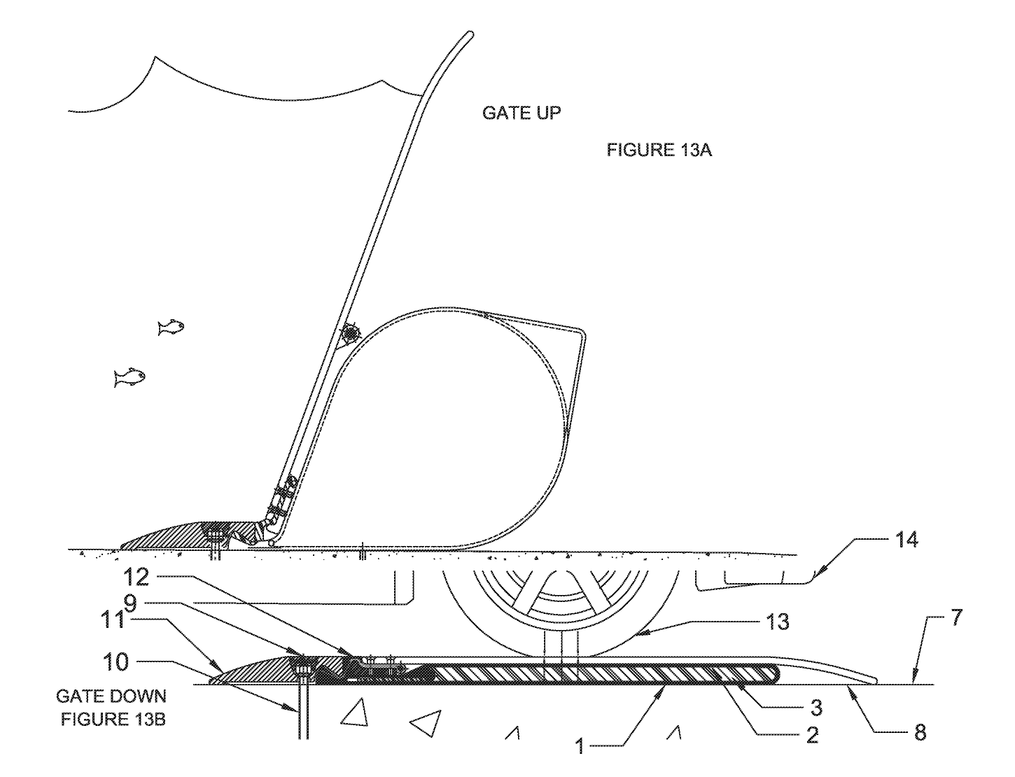

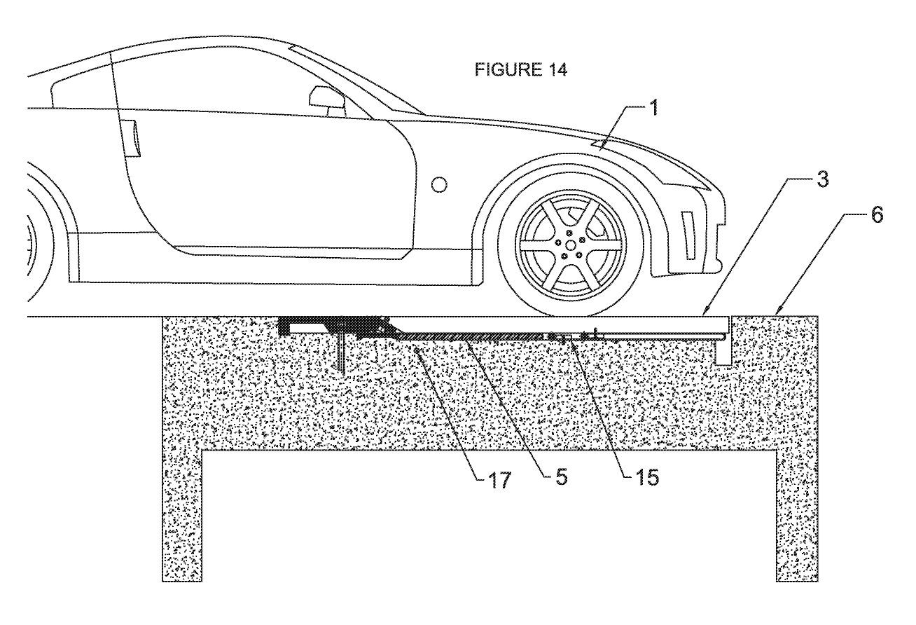

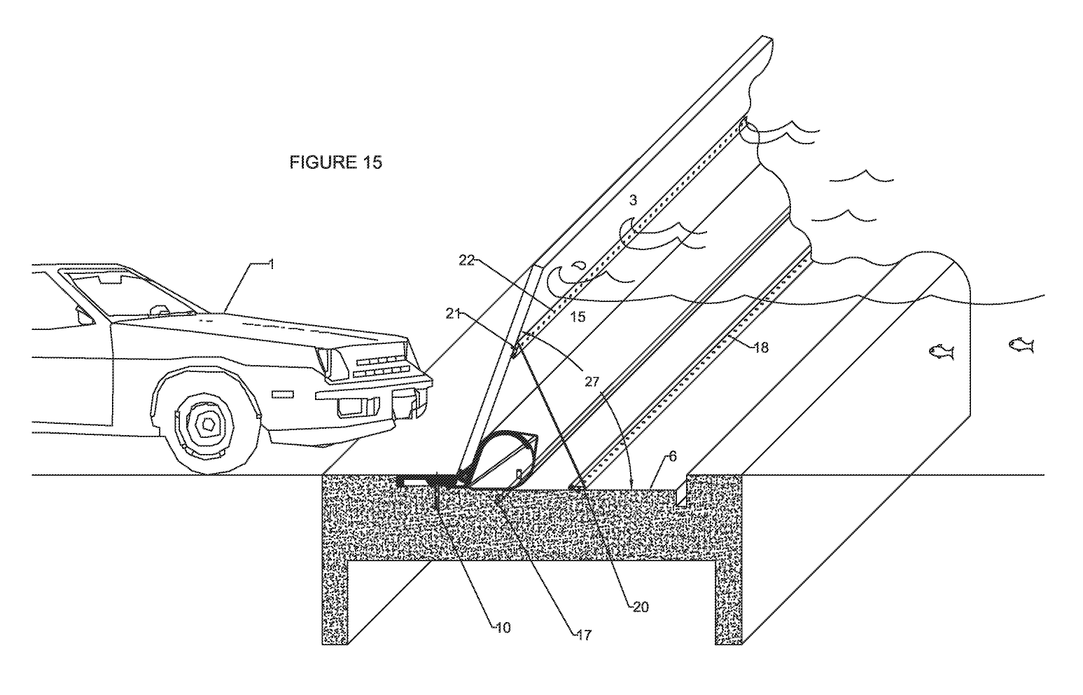

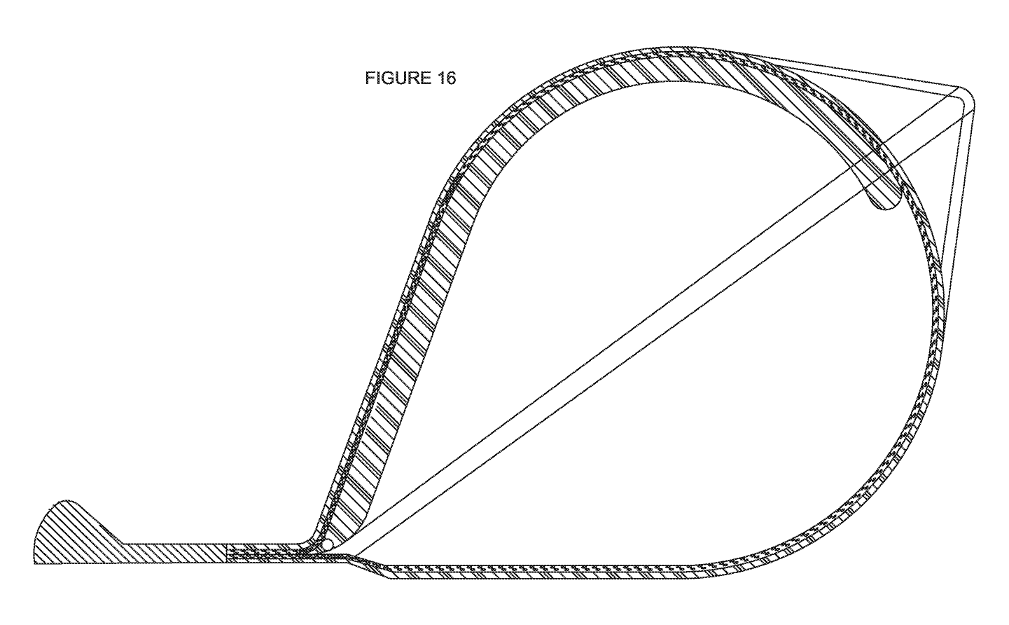

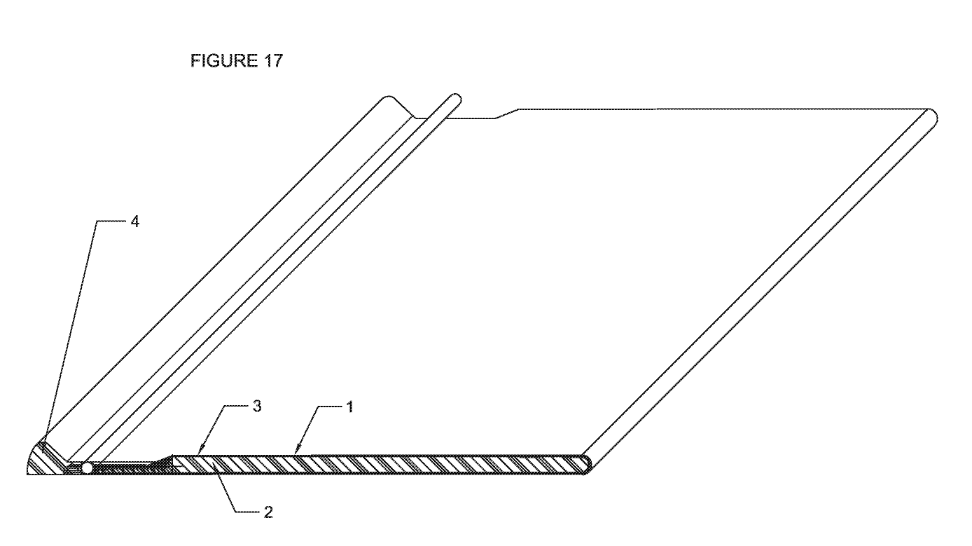

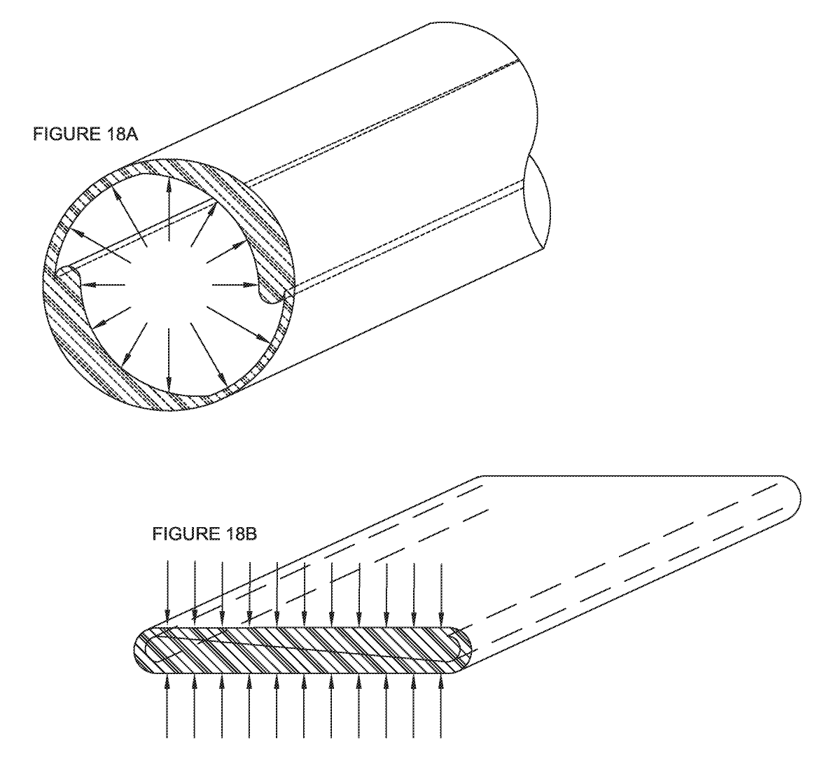

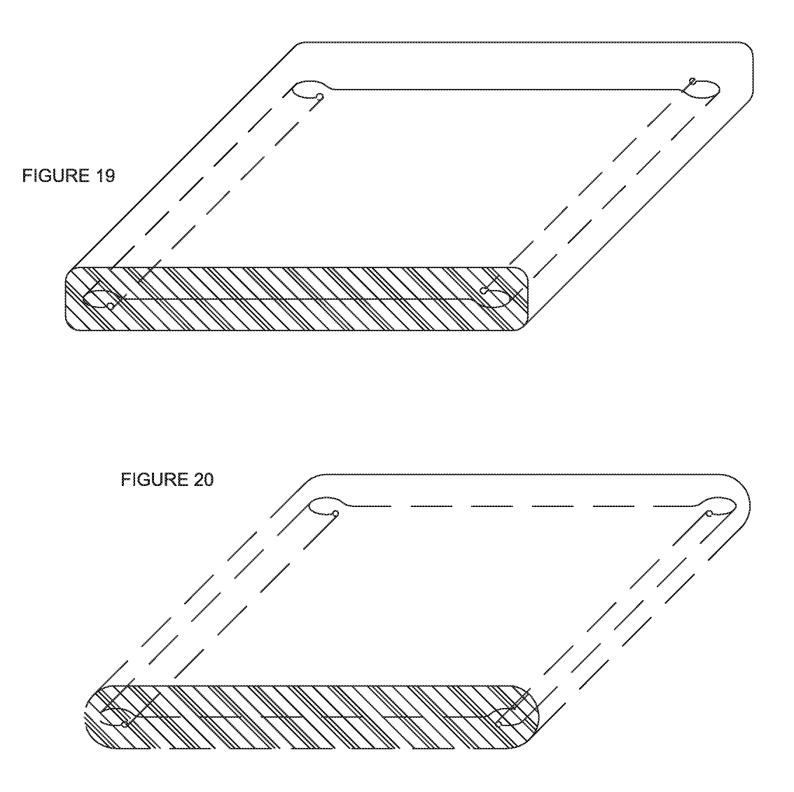

Referring to FIGS. 4a, 4b, 4c and 4 d, a hose is illustrated in its pressurized and depressurized configurations. Rubber mandrel 1 extends across the lower half of the circumference and along the length of the inflated hose of FIG. 4b and extends in planar form across the bottom half of and along the length of the depressurized hose of FIG. 4a. Rounded edges shown in FIGS. 4a and 4b of rubber mandrel support hose wall portion shown in FIGS. 4c and 4d against collapse under external pressure. Stress relief element 1 may be molded or extruded, for example, and cured or vulcanized prior to incorporation into the balance of the hose structure. In such case it may usefully serve as a mandrel on which the balance of the hose structure may be built and around which the balance of the hose structure may be cured. In accordance with prior art practice mandrel built hoses require removal of the mandrel after curing of the hose. In the case of small hoses lead or other low melting point metals, or solid salt may be used as mandrels later removed by melting or dissolution. Such methods are impractical for large diameter (500 mm diameter, for example) hoses, in which case steel tubes of lengths of perhaps 12 meters are used. The limited lengths of steel mandrels results in the need for closely spaced hose connections. Such hose connections are typically heavy, rigid, and expensive. A hose with a number of such connections is therefore ill-suited for efficiently coiling onto a spool. In accordance with at least one example of prior art, the hose body of circular cross section is itself is increased in diameter to match or exceed the hose coupling diameter so as to allow spooling of the hose with its couplings. Obviously such an arrangement provides a very poor ratio of hose length to spool size. Referring to FIG. 7, a hose in accordance with the present invention illustrated as it might be coiled onto a spool. Such a coiled hose is much more convenient to deploy than an assembly of many hoses which must be connected, sometimes under difficult conditions such as in a sub-sea environment.

The utility of the stress relief element 1 is not limited to mandrel built hoses. Extruded and unreinforced hoses may likewise be manufactured with a stress relief element incorporated into one half of the hose so as to limit both internal compressive forces and external tensile stresses within the structure of the unpressurized or externally pressurized hose and to prevent high internal tensile stresses on the interior of the pressurized hose that would otherwise occur in conjunction with high compressive stresses along the exterior of the pressurized hose.

Hoses used for peristaltic pumps are subject to many cycles of alternating internal pressure and external compression. Hoses in accordance with the present invention, with a stress relief elements along one half of the interior, for example, in combination with a uniform exterior hose thickness are particularly suited for use in conjunction with peristaltic pumps. The uniform thickness attribute is especially convenient for peristaltic pumps designed for use with prior art hoses. However, in accordance with a further aspect of this invention, a peristaltic pump may be configured to use hose with stress relief inserts as elsewhere described in this application which hose need not necessarily be of uniform thickness in its collapsed configuration.

In accordance with a further embodiment of this invention, an elastomeric mandrel may be formed and cured, or partially cured, as an initial manufacturing step. Said elastomeric mandrel may be subsequently incorporated into an inflatable article such as a hose, tire, or pneumatic actuator, or into a storage article such as a tank of medical storage bag. One function of the elastomeric mandrel is to allow the structure of the inflatable article to be assembled and cured around the said elastomeric mandrel. The use of an elastomeric mandrel in accordance with the present invention enables articles that are rounded in shape when inflated or filled to be assembled, cured, and stored in a flat configuration. The need for removable or dissolvable interior mandrels is thus eliminated.

A consequent desirable characteristic of articles which are cured in a flattened configuration is that they inherently return to a flat configuration when empty or deflated. Such a flat configuration is generally easier to store and transport.

Additionally, the flattened configuration results in thorough emptying of the article, this being an advantage in the case of valuable liquids or liquids that might result in spoilage, contamination, fire hazard, excess weight, or pollution if left in place. In the case of hoses, residual liquid can result in the inability of the hose to be tightly wound.

In accordance with a further aspect of the present invention, a hose or other elastomeric article may be constructed of alternating layers of orthotropically strain oriented polymer. In this manner, a structure may be created that can readily strain in shear to desirably distribute loads across the structure.

In accordance with a further aspect of this invention, strain oriented polymer may be oriented normal to a wear surface for maximization of wear life. The resulting structure results in long polymer chains being directly anchored into the main body of the structure, such as a tire, as opposed to being largely dependant on cross linking to resist wear.

In accordance with a further aspect of this invention, a removable mandrel portion, of cylindrical form, for example, may be used in conjunction with an elastomeric mandrel in order to establish an inflation channel and to facilitate the sequential connection of multiple actuators or storage articles. Such an arrangement is advantageous for actuators for flood control gates that must be installed on a street or on a parking garage ramp, for example, where installation of embedded pipes in the roadway would be expensive and inconvenient. Such an arrangement also eliminates the need for air connection blackouts in a roadway.

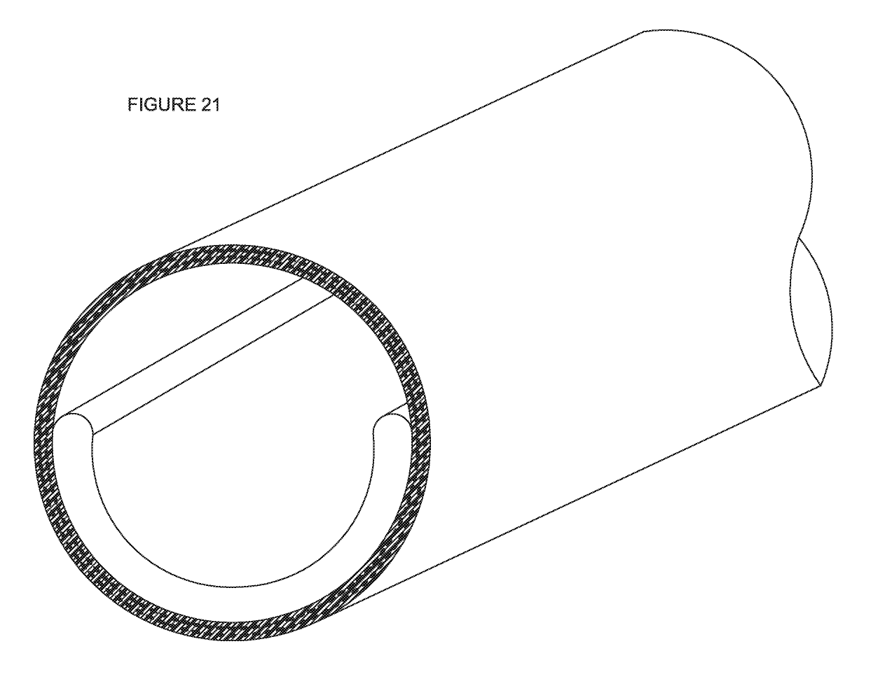

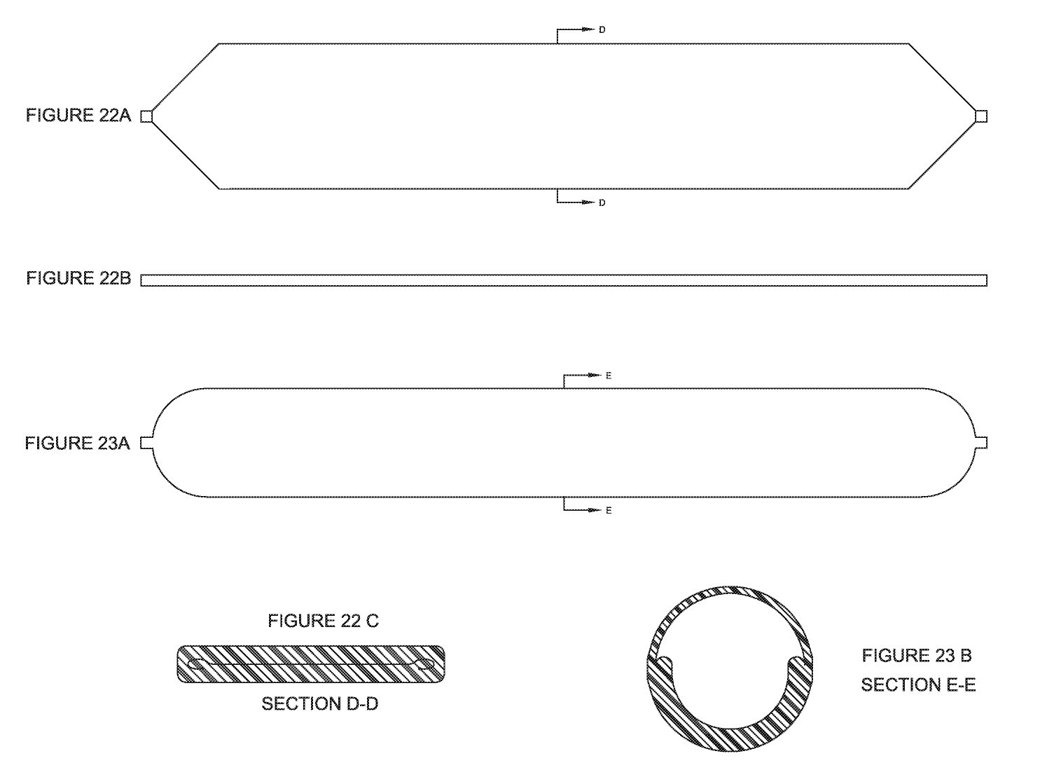

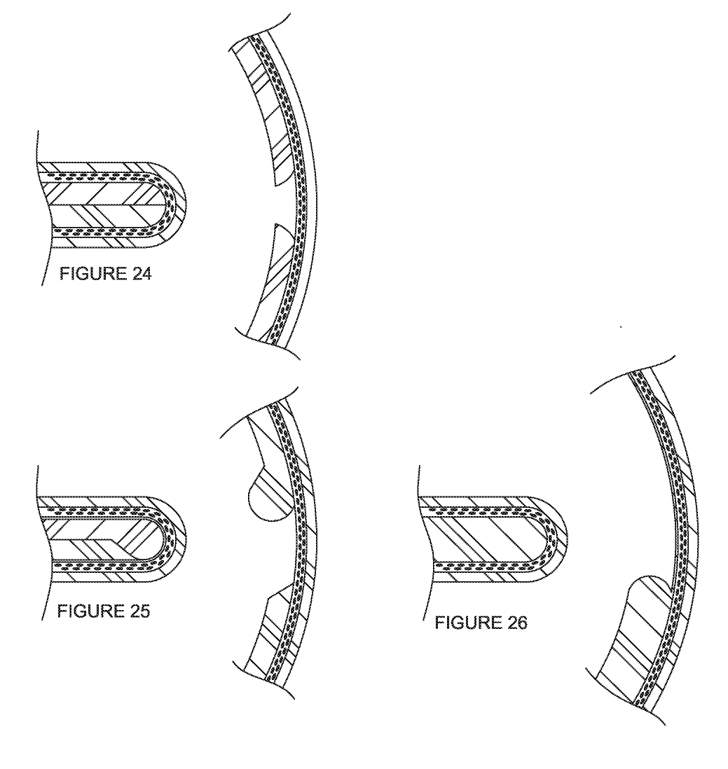

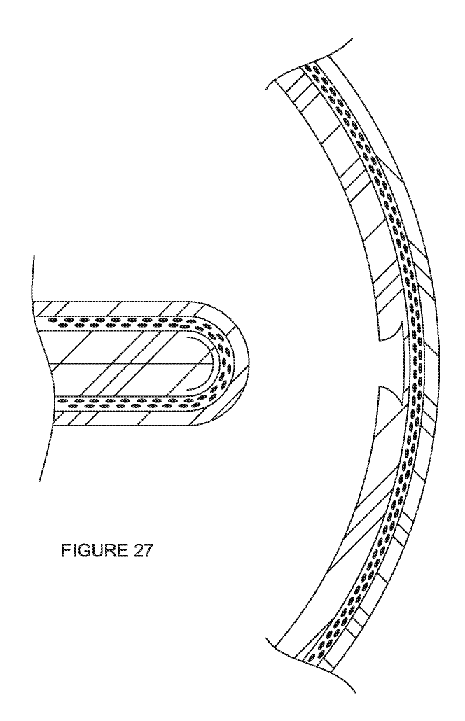

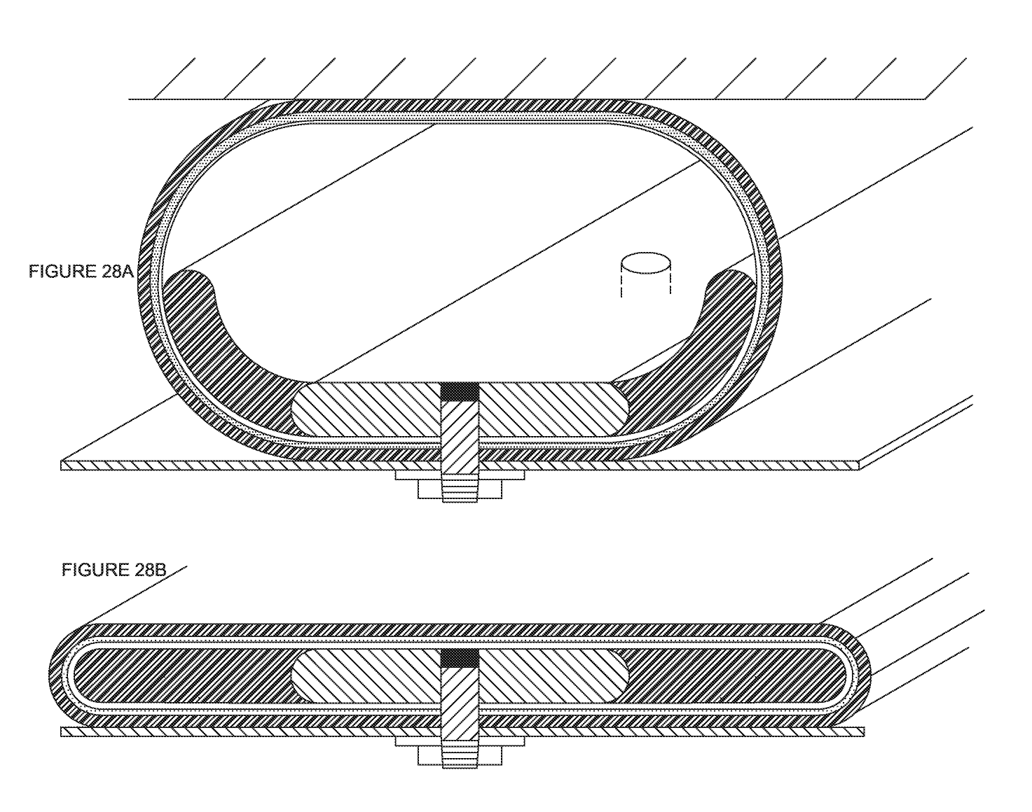

Referring to FIGS. 21 and 22, gate panel 3 is deployed by pressurizing air bladder 5 through air fitting 26 with air through inflation pipe 17. Gate panel 3 is maintained in position against hydrostatic pressure and wave pressure by membrane 15. Membrane 15 may be discontinuous, or may be a continuous member. A continuous member may usefully prevent surf-borne debris from impact and possibly puncturing air bladders 5. The angle 27 of the gate panel is preferably less than 90 degrees in order that waves are deflected back toward their source.

1. An inflatable article comprised of an inflatable envelope and an internal elastomeric mandrel with at least one round edge, wherein the inflatable envelope and the internal elastomeric mandrel are bonded together over a portion of one face sufficient to secure the mandrel in a fixed position relative to the inflatable envelope during repeated inflated inflate and deflate cycles, and wherein said inflatable envelope is not bonded to the at least one round edge of the internal elastomeric mandrel.

2. The inflatable article of claim 1, wherein the inflatable article is a hose.

3. The inflatable article of claim 1 wherein the inflatable article is a pneumatic actuator for a spillway gate.

4. The inflatable article of claim 1; wherein the inflatable article is a toroidal tunnel seal.

5. The inflatable article of claim 1, wherein the inflatable article is a consumer device for flotation, water rescue or human comfort.

6. An elastomeric article comprised of a plurality of strain oriented layers plied together sequentially so as to obtain desired directional physical properties.

7. An elastomeric membrane comprised of a plurality of strain oriented layers of elastomeric compound plied together in alternating directions so as to obtain orthotropic elastic properties.

8. The elastomeric membrane of claim 6 utilized as an inflatable article or portion thereof.

9. The elastomeric membrane of claim 6 configured as a hose or portion thereof.

10. The membrane of claim 9 utilized as a spinal disk prosthesis or portion thereof.

11. An elastomeric membrane locally defining an x-y plane comprised of a plurality of strain oriented layers of elastomeric compound plied together so as to establish a predominately z axis orientation of the constituent polymer molecules.

12. The membrane of claim 9 utilized as a wear surface.

13. The membrane of claim 9 utilized as a friction surface.

14. The membrane of claim 9 utilized as the tread of a tire.

15. The membrane of claim 9 utilized as the wear surface of a power transmission belt.

16. The membrane of claim 9, utilized as the wear surface of footwear.

17. A water control gate deployed by one or more inflatable actuators and restrained against hydrostatic forces by restraining means separate from said inflatable actuator.
